(19) United States
(12) Patent Application Publication
(10) Pub. No.: US 2016/0370338 A1
(43) Pub. Date: Dec. 22, 2016

(54) SENSOR DATA PROCESSING IN A DISTRIBUTED SENSOR SYSTEM

(71) Applicant: Aclima Inc., San Francisco, CA (US)

(72) Inventor: Gigi Sayfan, Davis, CA (US)

(21) Appl. No.: 14/746,400

(22) Filed: Jun. 22, 2015

(51) Int. Cl. G01N 33/00

(52) U.S. Cl. CPC G01N 33/0062; G01N 33/0008

(57) ABSTRACT

A sensor data processing system and method is implemented in a distributed sensor system to facilitate the collection of sensor data generated from multiple sensors deployed in the field. The sensor data processing system and method processes the large volume of sensor data to generate sensor data with high data quality. More specifically, the sensor data processing system is implemented as a generically defined, metadata driven system to allow the distributed sensor system to be expanded or modified with ease. Furthermore, the sensor data processing system and method applies backend calibration to the sensor data to enhance the accuracy of the sensor data. Finally, the sensor data processing system and method of the present invention implements parallel sensor data processing using a processing pipeline to process each sensor data message for scalability and increased reliability.

### BACKGROUND OF THE INVENTION

Environmental monitoring, sensing and measurements have become of interest to enterprises and individuals wishing to assess the current conditions of the environment in which individuals live or work or to characterize and monitor the quality of the environment. In some cases, environmental monitoring is performed to establish trends in environmental parameters, such as for monitoring operations of building heating and cooling systems or operation of factory machinery. In other cases, environmental monitoring may be performed for detecting climate changes. Environment sensing and measurements can include air quality monitoring, water quality monitoring, and monitoring of other environmental factors.

Air quality is a measure of the condition of air relative to the requirements of human need or purpose. Outdoor air quality monitoring is performed to measure the levels of pollutants in the air so as to detect potential harmful air pollution. Outdoor air quality monitoring is typically carried out using monitoring stations installed at various physical locations. These monitoring stations measure the presence of contaminants in the air, such as carbon monoxide, ozone, particulate matter, sulphur dioxide (SO₂) and carbon dioxide (CO₂). Indoor air quality monitoring is becoming a matter of interest as the air in enclosed spaces, such as home, schools or workplaces, can also be polluted. Conventional air quality monitors are expensive and require complex calibration procedure to ensure accurate measurements.

### BRIEF DESCRIPTION OF THE DRAWINGS

Various embodiments of the invention are disclosed in the following detailed description and the accompanying drawings.

FIG. 1 is a system diagram illustrating an embodiment of a distributed sensor system for measuring air quality in an environment.

FIG. 2 is a block diagram illustrating a data collection engine in the centralized backend system of the distributed sensor system in embodiments of the present invention.

FIG. 3 illustrates the data processor configured for parallel processing in the sensor data processing system in embodiments of the present invention.

FIG. 4 illustrates a sensor data message in embodiments of the present invention.

FIG. 5 is a flowchart illustrating a sensor data processing method in embodiments of the present invention.

FIG. 6 illustrates an example sensor data record in the sensor data database in embodiments of the present invention.

FIG. 7 illustrates a configuration of metadata tables in the metadata database to enable the generically configured and metadata driven system in embodiments of the present invention.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

A sensor data processing system and method is implemented in a distributed sensor system to facilitate the collection of sensor data generated from multiple sensors deployed in the field. The distributed sensor system often includes a large amount of sensors deployed over a wide geographic area, including stationary sensor installations or mobile sensor deployment. The sensors communicate with a centralized backend system over a data network and transmit continuous real-time sensor data to the centralized backend system. In some embodiments, the centralized backend system implements the sensor data processing system and method of the present invention to receive and process the continuous real-time sensor data from multiple data sources. The sensor data processing system and method processes the large volume of sensor data to generate sensor data with high data quality. More specifically, the sensor data processing system is implemented as a generically defined, metadata driven system to allow the distributed sensor system to be expanded or modified with ease. The sensor data processing system can be readily adapted to new configurations or new elements in the distributed sensor system.

For instance, the sensor data processing system and method applies backend sensor-specific calibration or compensation to the sensor data to enhance the accuracy of the sensor data based on factors associated with the sensing environment, aging of the sensors and drifting of sensor accuracy over time. The sensor-specific calibration is performed at the centralized backend system and not at the individual sensor module. In this manner, the cost and complexity of implementing the distributed sensor network are greatly reduced. Meanwhile, the sensor data quality is ensured by the sensor data processing system and method of the present invention implemented at the centralized backend system. Furthermore, the sensor data processing system and method stores the sensor data to enable fast query of the sensor data. In some embodiments, the sensor data is stored using multi-level aggregation that allows the sensor data to be queried efficiently and quickly.

Furthermore, in embodiments, the sensor data processing system and method of the present invention implements parallel sensor data processing for scalability and increased reliability. Using parallel data processing, the sensor data processing system can be scaled up easily as additional sensors are deployed in the distributed sensor system and the volume of sensor data increases. Thus, the distributed sensor system can be expanded by deployment of additional sensors and the sensor data processing system can be readily scaled up to process the increased volume of continuous real-time sensor data.

Distributed Sensor System

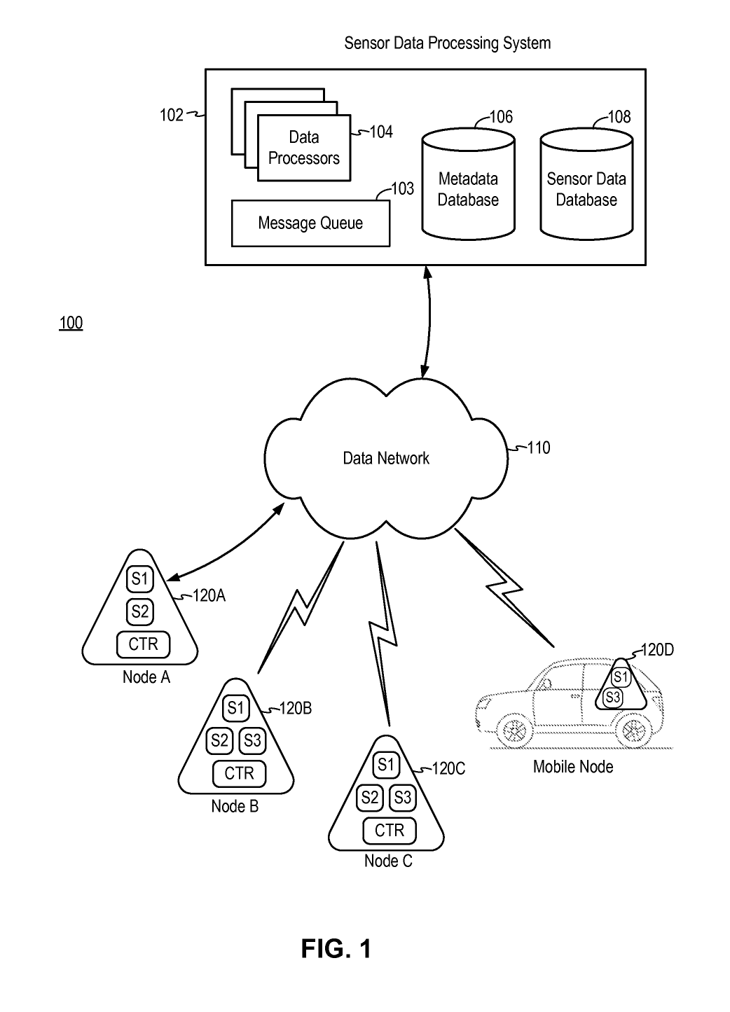

FIG. 1 is a system diagram illustrating an embodiment of a distributed sensor system for measuring air quality in an environment. An example of the distributed sensor system is also described in copending and commonly assigned U.S. patent application Ser. No. 13/800,172, filed Mar. 13, 2013, entitled “Distributed Sensor System With Remote Sensor Nodes And Centralized Data Processing,” of Risk et al, which application is incorporated herein by reference in its entirety. Referring to FIG. 1, a distributed sensor system 100 including a set of spatially distributed base units 120, also referred to as “sensor nodes” or “nodes,” configured to obtain measurements from the environment. In FIG. 1, sensor nodes A to D (120A to 120D) includes fixed base units 120A-120C installed at various physical locations and a mobile base unit 120D installed on a vehicle. Alternately, a mobile base unit can also be affixed to a person as a wearable sensor node or affixed to a vehicle as a mobile sensor node. The sensor nodes in system 100 may be deployed in an indoor location or an outdoor location or both for monitoring the local air quality. Alternately, the sensor nodes in system 100 may be deployed on a vehicle for monitoring the air quality while traveling in an area. In some embodiments, a set of sensor nodes are strategically deployed in a location to obtain sufficient amount of measurements to assess the air quality in a location. For example, in one embodiment, a set of sensor nodes are spatially dispersed in a building, such as a workplace or a factory, to monitor the air quality in the building. In other embodiments, a mobile base unit on a vehicle may be driven to traverse an area to obtain air quality measurements over a geographic area.

Each base unit 120 includes one or more sensor modules (S#) and a controller (CTR) which incorporates therein a transceiver. A sensor module S# incorporates a sensor for detecting a specific air quality parameter. The sensor modules may include different types of sensors for sensing different air quality parameters. A set of base units 120 deployed in an installation may be configured with sensor modules with the same sensor type. For example, nodes A, B, C and D all include sensor module S1. A set of base units 120 may also be configured with sensor modules with different sensor types. For example, nodes B, C and D include the sensor module S3 and not Node A. Furthermore, the mobile sensor node 120D may include one or more sensor modules that are the same or different from the sensor modules in a fixed base unit.

A salient feature of the base unit 120 in the distributed sensor system of the present invention is that the base unit is configurable to allow any desired types of sensors to be incorporated for measuring the desired environment parameters. Thus, a set of nodes for an installation can be configured with the same set of sensor modules to measure the same set of air quality parameters. Alternately, a set of nodes for an installation can be configured with different sensor modules to measure a different set of air quality parameters at each node. One or more sets of mobile sensor nodes may also be equipped with the same or different set of sensor modules. In one example, the types of sensors that can be included in a base unit include CO₂, O₂, CO, CH₂O, NO₂, NO, O₃, CH₄, Particulate Matter, Volatile Organic Compound (VOC), Humidity, Light, Temperature, Sound, Pressure and Vibration.

Each of the base units 120 includes a transceiver to communicate with a centralized backend system 102 through a data network 110. The base units 120 may employ wired communication, such as Ethernet, or wireless communications, such as radio frequency (RF), WiFi, ZigBee or other low power short-range wireless communication protocols. The controller CTR controls the sensing and communication functions of the base unit. The data network 110 may include a private data network, the public Internet, or a combination of both.

In embodiments of the present invention, the centralized backend system 102 functions as the sensor data processing system. The centralized backend system 102 may be implemented using one or more computing systems or one or more server appliances. Implementing the centralized backend system 102 using multiple server appliances improves the reliability of the system. In the present description, the term “centralized backend system” refers to one or more computing units, which can be physical or logical, for performing the data processing and storage functions described herein where the centralized backend system can be implemented using one or more server appliances. In some embodiments, the centralized backend system 102 is implemented as a distributed server system and may include servers of different server types, such as physical or logical servers. The centralized backend system 102 may be distributed over multiple physical and/or virtual machines and also may be distributed geographically over a large area, including over the globe. It is instructive to note that while the base units 120 sends the sensor data logically to one destination—the centralized backend system 102, the sensor data may be processed and stored in a distributed manner in the servers forming the centralized backend system.

In the distributed sensor network 100, the network of base units 120 may include a large number of sensors deployed in an environment where the sensors are operative to generate massive amount of sensor data in real-time. The sensor data are fed back to the centralized backend system 102 for processing and storage. The centralized backend system must be capable of receiving and processing the massive amount of sensor data arriving in real-time. Furthermore, the sensor data should be processed in a way that guarantees sensor data quality. High quality sensor data means that the processed sensor data gathered from the deployment of sensors are accurate and meaningful. Conventional distributed sensor systems typically cannot provide high sensor data quality due to large calibration errors that may be present at the sensors, aging of sensors resulting in drifting of the sensor's measurement accuracy and sensing abnormality that may be introduced by the sensing environment.

In embodiments of the present invention, the centralized backend system 102 implements the sensor data processing system and method to handle the large amount of incoming real-time sensor data arriving in real-time. More importantly, the sensor data processing system and method ensures high sensor data quality and absence of sensing data errors due to calibration or other sensor limitations. In the present description, the centralized backend system 102 will sometimes be referred to as the sensor data processing system 102.

In embodiments of the present invention, sensor data processing system 102 includes a message queue 103, a data processor 104, a sensor metadata database 106 stored in a computer data storage or memory, and a sensor data database 108 stored in a computer data storage or memory. The sensor metadata database 106 stores management data or metadata associated with each sensor node in system 100. In some embodiments, the metadata includes identification information for the sensor node, such as a Node Identifier or a serial number. In other embodiments, the metadata also includes calibration data for the sensor in each base unit. In accordance with embodiments of the present invention, the sensor metadata database stores metadata tables which are used to construct the definitions for the data tables in the sensor data database. In this manner, the sensor data database can be easily updated to respond to changes in the sensor system.

In operation, the sensor data processing system 102 stores in the sensor data database 108 raw sensor data received from the base units 120 over the data network 110. Data processor 104 is configured to process the raw sensor data to convert the sensor data to meaningful physical units and to calibrate the sensor data using the calibration data stored in the sensor metadata database 106 to generate calibrated sensor data which can then be used to assess the air quality at the installation where sensor nodes 120 are deployed. The calibrated sensor data may also be stored in the sensor data database 108. The calibrated sensor data, as well as the raw sensor data, may be accessible through one or more application programming interface (API) to allow users to monitor the air quality measurements obtained by the distributed sensor system 100.

A feature of the distributed sensor system of the present invention is that each sensor module provides raw sensor data to the sensor data processing system to be stored and processed. In the present description, raw sensor data refers to sensor data that has not been modified or calibrated based on performance characteristics of the particular sensor that generated the sensor data. Most sensors have certain amount of non-linearity characteristics over time and sensors need to be calibrated for the non-linearity or performance drift over time. Typically, a sensor may have gain or offset that drifts over time as the sensor is being used in an environment.

Conventional sensors are often calibrated periodically, such as annually, and the calibration data, such as a gain correction value and an offset correction value, for the sensor is stored with the sensor itself and sensor data is modified using the calibration data as the sensor data is being generated by the sensor. In the conventional sensors, when a sensor's characteristics drift over time before the next calibration update, the calibration data may no longer be accurate for that sensor. However, the sensor will continue to use the inaccurate calibration data to calibrate or modify the sensor data. Thus, conventional sensors may end up generating sensor data that has embedded calibration error and the sensor data is permanently corrupted. In embodiments of the present invention, the distributed sensor system 100 implements a centralized backend calibration method where the base unit 120 reports raw sensor data that have not been modified to the centralized backend system 102. Calibration of the raw sensor data is performed at the centralized backend system 102 to generate calibrated sensor data using calibration data stored at the centralized backend system. The availability of the raw sensor data allows the centralized backend system to generate calibrated sensor data using updated or corrected calibration data so that accuracy of the sensor measurement can be assured. More importantly, when calibration data for a sensor is found to be inaccurate later on and new calibration data is generated, the centralized backend system may regenerate the calibrated sensor data by retrieving the raw sensor data for that sensor and calibrating the raw sensor data again using updated calibration data. In this manner, historic sensor data can be corrected if the calibration data used was found to be inaccurate. Correction of historic sensor data is not possible in conventional sensors because the calibration was done at the sensor and raw sensor data is typically not available. The centralized backend calibration method in the distributed sensor system of the present invention is described in copending and commonly assigned U.S. patent application Ser. No. 13/800,199, filed Mar. 13, 2013, entitled “Calibration Method For Distributed Sensor System,” of Herzl et al., which application is incorporated herein by reference in its entirety.

In FIG. 1, the sensor calibration data (or sensor metadata) and the sensor data are shown as being stored in two databases. The two databases can be two physical databases or two logical databases. The exact configuration of the two databases is not critical to the practice of the present invention. The storage of the sensor metadata and the sensor data can be made in one physical data storage or multiple physical data storages.

Sensor Data Processing System

In the distributed sensor system 100, sensor data are being generated at a high data rate from each sensor deployed in the installation. For example, sensor data is generated every second. In a typical installation, a large number of sensor nodes, each with multiple sensors, may be deployed. Thus, the distributed sensor system 100 generates a high volume of data at a high data rate. For example, for an installation with 500 nodes each with 12 sensors, a total of 6000 sensors are deployed in the system. When each sensor generates sensor data every second, 6000 sensor messages are being generated every second. The sensor data processing system and method of the present invention is configured to receive and process the high volume of sensor data generated at the high data rate to support the operation of the distributed sensor system 100.

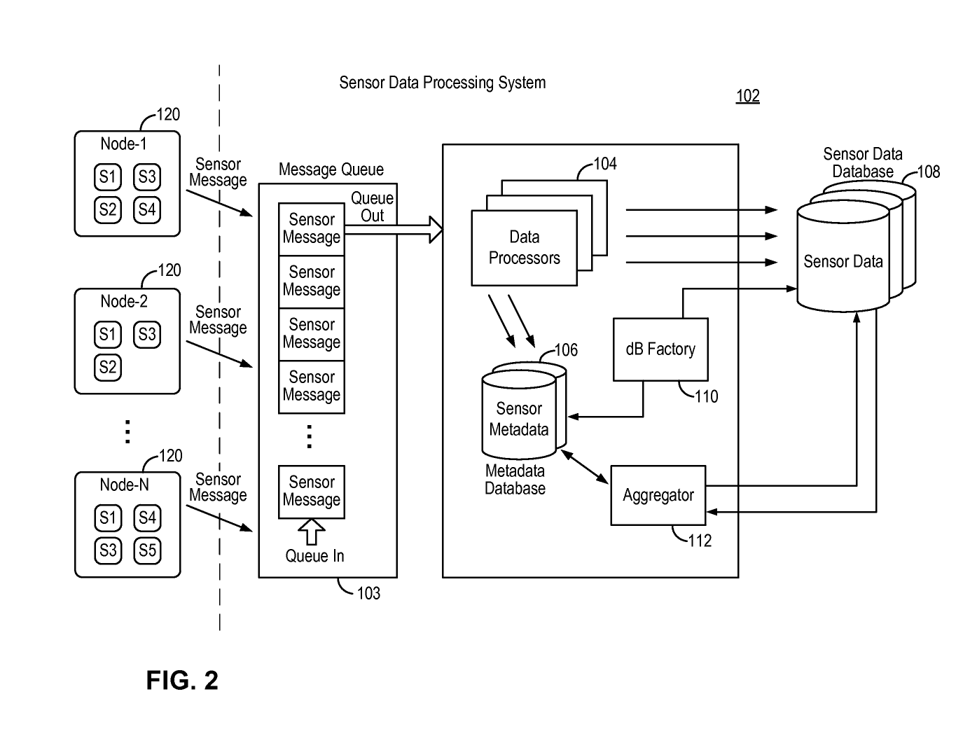

FIG. 2 is a block diagram illustrating a sensor data processing system implemented in the centralized backend system of the distributed sensor system in embodiments of the present invention. Referring to FIG. 2, in the distributed sensor system 100, a set of sensor nodes 120 is deployed in an installation to measure air quality parameters. The sensor nodes 120 may be deployed within a building or over several buildings or in a vehicle. In some examples, the sensor nodes 120 may be deployed in a corporation's workplaces in several countries over several continents. Each sensor node 120 includes one or more sensor modules containing a sensor. A sensor module or sensor (S#) is provided for detecting a specific air quality parameter. Additional sensors may be included for measuring environmental qualities, such as sound, temperature, humidity, pressure and ambient light condition. In the present example, the sensors nodes Node-1 to Node-N may include different type of sensors to sense different air quality parameters and environmental parameters. Each sensor node 120 is configured to send sensor messages to the centralized backend system 102 through the data network 110. The sensor messages may include sensor management messages and sensor data messages. Sensor management messages may be sent for set up and configuration of the sensor module and the sensor node. The sensor data processing system and method of the present invention is concerned with receiving and processing the sensor data messages that are sent by each sensor module to report sensor readings. In the present description, sensor data messages will also be referred to as “sensor messages” and it is understood the sensor messages referred herein contain sensor data instead of management or configuration data and information.

In embodiments of the present invention, each sensor in a sensor node 120 sends a periodic sensor message to report the sensor's recent readings. The sensor data message includes identifying information for the sensor such as a Node identifier (Node ID) identifying the sensor node, a board identifier (Board ID) identifying the sensor board in the sensor node, and a sensor identifier (Sensor ID) identifying each sensor in a sensor node. In some embodiments, the identifying information may further include a sensor type identifier (Sensor Type ID) to identify the type of sensor (e.g. CO₂, O₂ or humidity). In some cases, the separate Sensor Type ID is not needed and the Sensor ID provides the identification of the type of sensor as well as the specific sensor reporting sensor data.

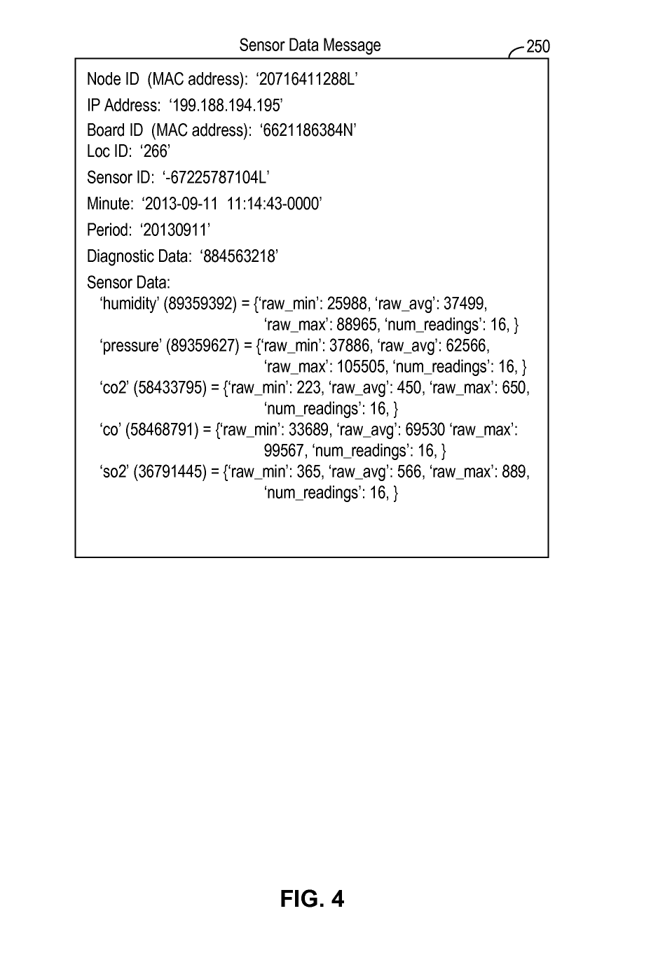

In embodiments of the present invention, a sensor node sends a periodic sensor message to report the sensor readings from one or more sensors of the sensor node. The sensor readings can be air quality measurements or environmental measurements. FIG. 4 illustrates a sensor data message in embodiments of the present invention. Referring to FIG. 4, a sensor message 250 includes identifying information such as a Node identifier (Node ID) which can be the MAC address of the sensor node, a Board identifier (Board ID) which can be the MAC address of the sensor board in the base unit, and an IP address. From the Node ID and the Board ID, a location identifier (Loc ID) and a sensor identifier (Sensor ID) are derived for each sensor reading. In the case where the sensor data message is from a reference equipment or an equipment used for calibration, the Board ID can be the serial number of the reference/calibration equipment. The sensor message 205 further includes the timestamp of the sensor readings being reported. For example, the timestamp can be expressed in the form of the date and time in terms of the minute in which the sensor readings are generated. For example, a Minute timestamp may be given in the form of the year, month, day and the time in minute in which the sensor readings are generated. In some cases, additional timestamp indicator may be used. For example, a Period timestamp may be used to indicate the time period in which the sensor readings are generated.

In some embodiments, the sensor data message may also contain diagnostic data for the sensor node. The diagnostic data may include an error code or operation condition of the sensor node. The centralized backend system may use the diagnostic data to analyze the health of the sensor node and to detect for system issues, if any, associated with the sensor.

In the embodiment shown in FIG. 4, the sensor data message 250 includes sensing readings from several air quality sensors and also several environmental sensors. In the present example, sensor readings are reported from sensors for humidity, pressure, CO₂, CO and SO₂. The sensor data message includes a set of raw sensor data values for each sensor. In the present example, each sensor is configured to report sensor data on a periodic basis, referred herein as a reporting interval or a reporting period. For example, the reporting period may be once per second or 1 Hz or once per minute or 0.02 Hz. However, in some cases, the sensor incorporated in a node may be capable of sampling the environment at a frequency greater than the reporting period. For example, while the reporting period may be 0.02 Hz or once per minute, the sensor may be capable of sampling at a rate of 60 Hz, that is, 60 times per second. In embodiments of the present invention, the sensor reports at each reporting interval the sensor readings collected during that interval. In the present example, the reporting interval is one minute and the sensor sampling rate is 16 times per minute. Accordingly, the sensor data message reports a set of raw sensor data values for each sensor including a maximum raw sensor reading, a minimum raw sensor reading, the average raw sensor reading of the sensor readings generated during the reporting interval, and the number of readings collected in that reporting interval. The sensor readings reported in the sensor data message are raw sensor data. The raw sensor data may be generated from analog or digital sensor readings which may be a current value or a voltage value. In most cases, the sensor module includes an analog-to-digital converter to digitize analog sensor readings to generate the raw sensor data values.

In the distributed sensor system 100, each sensor in each sensor node sends periodic sensor data messages to the centralized backend system 102 through the data network 100. Thus, the centralized backend system 102 receives a large amount of sensor data messages arriving at each reporting intervals, such as each second or each minute. Furthermore, in most implementations, the distributed sensor system will be deployed with a large number of installed sensor nodes and additional sensor nodes may be installed over time. Thus, the centralized backend system 102 should be capable of handling a large amount of incoming data and also be scalable so that additional processing resources can be added as more sensors are being installed. In embodiments of the present invention, the centralized backend system 102 implements the sensor data processing system of the present invention and uses a data processor that implements scalable parallel processing to handle the large amount of incoming sensor data messages. The parallel-processing data processor is scalable to allow the sensor data processing system to scale up as needed to handle any amount of incoming sensor data messages. In the embodiments of the present invention, the sensor data processing system includes software or hardware components or a combination of software and hardware components.

Returning to FIG. 2, the sensor data processing system 102 includes the message queue 103 for receiving and storing incoming sensor messages, the data processor 104 for processing the sensor messages, the sensor data database 108 for storing raw and processed sensor data, and the sensor metadata database 106 for storing metadata information relating to the sensor data. At sensor data processing system 102, sensor messages arriving at the sensor data processing system 102 are received into a message queue 103. In embodiments of the present invention, the message queue 103 is a memory or storage device implementing first-in-first-out operation. Accordingly, newly arriving sensor messages are received and stored at the end of the message queue and oldest sensor messages are retrieved and read out of the top of the message queue. The sensor messages read out of the message queue are provided to the data processor 104 for receiving and processing the sensor data messages.

In embodiments of the present invention, data processor 104 implements scalable parallel processing to handle the large volume of input data and to also enable the data collection system to be scaled up easily as additional sensors are deployed in the distributed sensor system. In some embodiments, data processor 104 is implemented using multiple parallel processing pipelines that are scalable and configurable. In some embodiments, the parallel processing pipelines are implemented as multiple software processes being executed on one or more microprocessors. In the present description, each processing pipeline is represented by a data processor 104 and the sensor data processing system 102 is illustrated as including a set of parallel data processors 104. In embodiments of the present invention, each data processor 104 performs the entire processing operation for a sensor data message and each data processor 104 is in communication with the sensor metadata database 106 to retrieve and store sensor metadata information and also in communication with the sensor data database 108 to retrieve and store raw and processed sensor data. Furthermore, while the multiple data processors may be executed on separate microprocessors, each data processor or each processing pipeline is configured to execute on a single microprocessor to ensure efficiency. Thus, the entire processing of a single sensor data message is handled by a single data processor running on a single microprocessor.

In embodiments of the present invention, the sensor data processing system uses the message queue 103 to ensure that each sensor message is processed and that sensor messages do not become lost or missing in processing chain. In operation, the message queue 103 stores incoming sensor messages and provides sensor messages to the data processors 104. Each sensor message remains stored in the message queue 103 until the data processor 104 indicates to the message queue 103 that the sensor message has been successfully processed. Only after an indication of successful processing is the sensor message being discarded from the message queue. In the event that a particular sensor message was not successfully processed, the sensor message remains in the message queue and will be sent out again to the data processor 104 for processing. In this manner, the message queue 103 ensures the reliability of the sensor data processing system and ensures that sensor data collected from the sensors in the distributed sensor system will be processed and not lost due to processing system mishaps.

The data processor 104 processes the sensor data messages by performing conversion and calibration operations to improve the sensor data quality. In particular, the sensor data processing system 102 is a generically configured, metadata driven system which uses generic data structure for the sensor data messages and uses metadata tables to define and configure the sensor data database. The sensor data processing system 102 is generically configured to allow processing of generic sensor data. As such, the sensor data processing system 102 implements a flexible platform for constructing the distributed sensor system. New sensors or new sensor types or new sensor modalities can be added to the distributed sensor system and the sensor data processing system can be easily configured to handle the new sensor data. In some embodiments, the sensor data messages are defined using generic data fields. Accordingly, the sensor data message can be readily adapted to any sensor type or modality. New sensor type or sensor modality can be added to the distributed sensor system without requiring any changes to the sensor data message for reporting the sensor data. In the present description, generic system configuration, generic data structure and generic data fields refer to configurations, data structures and data fields that are independent or agnostic to the sensor type or sensor modality. The same generic data structure and generic data fields can be applied to a wide range of sensor types and modalities.

The sensor data processing system 102 processes the sensor data messages using the sensor metadata information stored in the sensor metadata database 106. More specifically, the sensor metadata information includes identifying information for each sensor, such as the sensor identifier and the associated sensor type. The metadata information further includes calibration data associated with each sensor. The data processor 104 processes the raw sensor data in the generically defined data fields in the sensor data message using the metadata information to apply the correct conversion for the sensor data type and to apply the corresponding calibration data for that particular sensor to the sensor data.

In some embodiments, the sensor data processing system ensures high sensor data quality by performing centralized backend calibration using sensor-specific calibration data. As described above, the accuracy of a sensor tends to drift as the sensor age. The sensor may be calibrated periodically, such as by use of reference equipment or a reference sensor. The calibration data for the sensor is sent to the sensor data processing system 102 to be stored in the sensor metadata database 106. The data processor 104, when receiving a sensor data message for the particular sensor, retrieves the calibration data for that sensor from the sensor metadata database 106 to calibrate the raw sensor data values. In this manner, the sensor data processing system ensures high sensor data quality.

Furthermore, the sensor data processing system 102 uses the sensor metadata information to construct the definitions for the data tables in the sensor data database 108. In other words, the definition of data tables in the sensor data database 108 is metadata driven. Changes to the distributed sensor system which results in corresponding changes to the sensor data database, such as adding sensors or adding sensor types, can be implemented easily and quickly through changing just the metadata tables in the metadata database. It is instructive to note that the sensor data database 108 is typically a large database storing a large amount of data. Changing or updating such a large database is not easy. In embodiments of the present invention, the sensor data processing system uses metadata tables stored in the sensor metadata database 106 to construct the definition of the database tables in the sensor data database 108. Accordingly, changes to the sensor data database 108 can be implemented easily by merely updating or modifying the metadata tables in the sensor data metadata database 106.

In some embodiments, the sensor data processing system 102 includes a database (dB) factory 110 which queries the sensor metadata database 106 periodically for changes in any of the metadata tables. When a change in a metadata table is detected, the dB factory 110 sends instructions to the sensor data database 108 to create new data tables or to reconfigure the data tables in accordance with the detected changes in the metadata table. As thus constructed, any additive changes to the sensor data database 108 can be realized by merely making the change to the sensor metadata database 106. The sensor data database 108 can thus be readily reconfigured to accommodate changes in the distributed sensor system, such as to add a new sensor, new sensor type, a new geographic location or a new floor plan of an indoor structure. In embodiments of the present invention, the database factory 110 is a software process being executed on a processor.

In embodiments of the present invention, the sensor data processing system 102 further includes a sensor data aggregator 112 to generate aggregated sensor data for each sensor over one or more time intervals. In some embodiments, the sensor data aggregator 112 generates sensor data using multi-level aggregation to allow the sensor data to be queried efficiently and quickly. In operation, the sensor data aggregator 112 is in communication with the sensor data database 108 to receive raw sensor data and processed sensor data stored therein. The sensor data aggregator 112 retrieves the stored sensor data from the sensor data database 108 to generate aggregated sensor data and also stores the aggregated sensor data back into the sensor data database 108. In some embodiments, the time intervals for sensor data aggregation are metadata stored in sensor metadata database 106. The sensor data aggregator 112 communicates with the sensor metadata database 106 to obtain the time interval values for the desired sensor data aggregation. In this manner, the aggregation time intervals used by the sensor data aggregator 112 can be dynamically reconfigured by merely changing the values in the metadata database 106.

In some embodiments, the sensor data aggregator 112 performs sensor data aggregation at one or more low aggregation levels. For example, raw sensor data may be received from the sensors in the field on a 1-second interval and processed by the sensor data converter 154 at the 1-second interval. The sensor data aggregator 156 may aggregate the calibrated sensor data for each sensor over one or more low-level time intervals, such as a 1-minute interval and/or a 5-minute interval. In the present description, aggregation of sensor data of a sensor over a given time interval refers to averaging all of the sensor data of a sensor belonging to that time interval. In one example, when sensor data from a sensor is received on a 1-second interval, 1-minute sensor data may be generated by aggregating or averaging all the 1-second sensor data during each 1 minute interval. With aggregated sensor data thus generated at one or more low aggregation levels, the sensor data aggregator 112 can perform high-level sensor data aggregation using the low-level aggregated sensor data stored in the sensor data database 108. For example, 10-minute sensor data may be generated by aggregating or averaging the 1-minute sensor data during each 10 minute interval. In another example, 1-hour aggregated sensor data can be generated from the aggregated 10-minute data for the previous hour.

In some embodiments, the sensor data aggregator 112 implements the multi-level sensor data aggregation method described in copending and commonly assigned U.S. patent application Ser. No. 14/139,766, filed Dec. 23, 2013, entitled “Distributed Sensor Data Collection and Processing,” of Hu et al., which application is incorporated herein by reference in its entirety.

In embodiments of the present invention, the sensor metadata database 106 is implemented using a relational database, such as a SQL database. The relational database allows the sensor metadata information to be retrieved quickly when requested. On the other hand, in the embodiments of the present invention, the sensor data database 108 is implemented as a non-SQL (or NoSQL) database. The sensor data database 108 has to store a large amount of data and a non-relational or non-SQL database is more efficient. In the present description, a non-SQL or NoSQL database refers to a database that provides a mechanism for storage and retrieval of data that is modeled in means other than the tabular relations used in relational databases. In some embodiments, the sensor data database 108 is implemented using the Apache Cassandra™ database system being an open source distributed database management system designed to handle large amounts of data across many commodity servers.

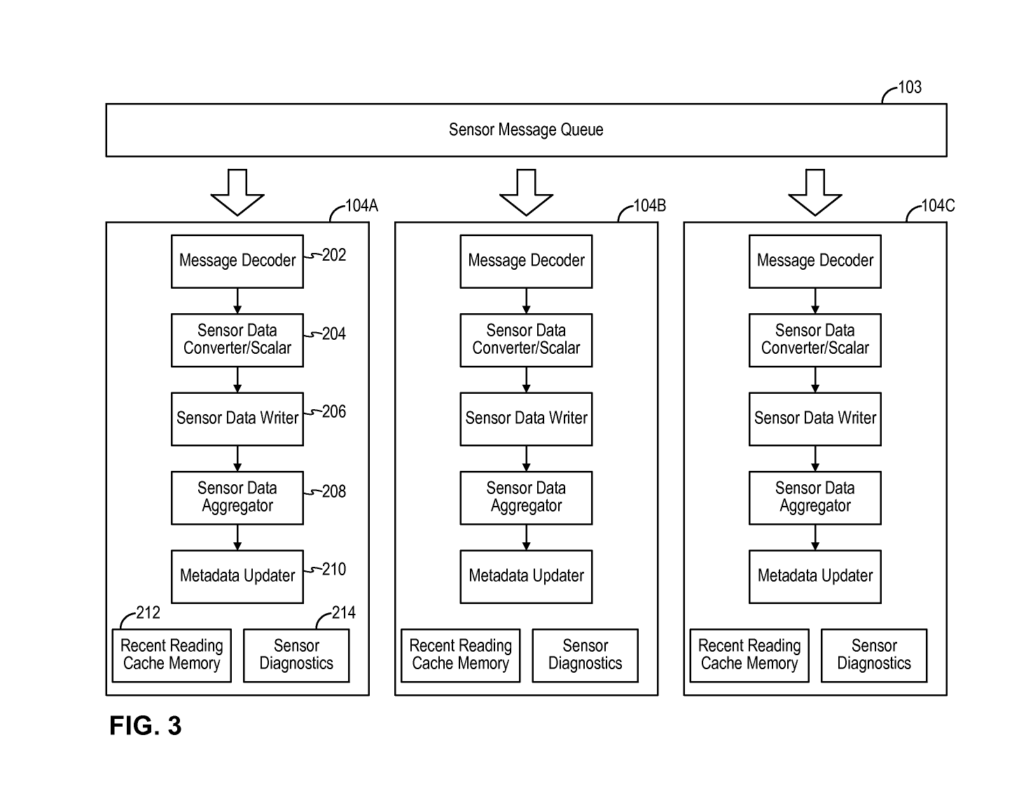

FIG. 3 illustrates the data processor configured for parallel processing in the sensor data processing system in embodiments of the present invention. Referring to FIG. 3, the data processor 104 of sensor data processing system 102 includes multiple parallel data processors 104A to 104C implementing multiple parallel processing pipelines. The data processor 104 is scalable and additional processing pipelines can be added as needed to scale up the data processor to increase the processing capacity. In the present description, the term “data processor” and “processing pipeline” are sometimes used interchangeably to refer to the data processor for processing sensor data messages. In the present embodiment, three data processors 104A to 104C are shown for illustration only and are not intended to be limiting. In actual implementation, data processor 104 will include multiple parallel data processors to handle the sensor data messages from message queue 103.

Each data processor 104 includes configurable processing stages or modules forming the processing pipeline. In some embodiments, each data processor 104 includes plug-in processing stages or modules that are configurable. That is, the processing stages in the data processor 104 may be configured by modifying or replacing one or more plug-in processing stages. In some embodiments, additional plug-in processing stages are added to the data processor 104 to increase the sensor data processing capabilities of the centralized backend system. For example, additional plug-ins may be added to introduce additional data processing operations. In embodiments of the present invention, each data processor 104 is executed on the same microprocessor. The multiple data processor 104 may be executed on separate microprocessors but the processing stages of a single processing pipeline are executed on the same microprocessor. Executing the processing stages of a single processing pipeline on the same microprocessor ensures efficiency.

In the sensor data processing system 102, the sensor message queue 103 feeds sensor messages to the data processors 104A to 104C for processing. Each data processor 104 is configured to decode, convert and calibrate the sensor data in the sensor data message. More specifically, the sensor message queue 103 provides a sensor data message to each of data processors 104A to 104C. The sensor data messages provided to the data processors persist in the sensor message queue 103 until the sensor message is processed successfully by the processing pipeline. After the sensor message queue 103 receives an indication from the data processor 104 that a particular sensor message has been successfully processed, that sensor message is then discarded from the sensor message queue.

In embodiments of the present invention, each data processor 104 includes the following processing stages: a message decoder 202, a sensor data converter scaler 204, a sensor data writer 206, a sensor data aggregator 208 and a metadata updater module 210. Each data processor 104 may further include a recent reading cache memory 212 and a sensor diagnostics module 214. It is instructive to note that the processing stages of the data processor 104 are configurable and additional stages can be added in or stages can be removed or modified.

The message decoder 202 performs decoding of the sensor data messages to obtain the identifying information and the raw sensor data from the sensor data message.

The sensor data converter scaler 204 processes the decoded raw sensor data. The sensor data converter scaler 204 is in communication with the sensor metadata database 106 to receive calibration data for the subject sensor. More specifically, the sensor data converter scaler 204 processes the raw sensor data to convert the sensor readings to meaningful physical units and also to calibrate the raw sensor data using the calibration data for that sensor retrieved from the sensor metadata database 106. In some embodiments, the sensor data converter scaler 204 performs the sensor data conversion and sensor data calibration as separate computation steps. In other embodiments, the sensor data converter scaler 204 performs the sensor data conversion and sensor data calibration in a single computation step. The exact configuration or the exact number of computation steps used in performing the sensor data conversion and sensor data calibration is not critical to the practice of the present invention.

The sensor data writer 206 is in communication with the sensor data database 108 to send the decoded raw sensor data with the identify information (such as Node ID and Sensor ID) to the sensor data database 108 for storage. The sensor data writer 206 also sends the calibrated sensor data to the sensor data database 108 for storage. In some embodiments, the raw sensor data and the calibrated sensor data are stored in the sensor data database 108 in the same document. For instance, the raw sensor data is typically expressed as a numeric string. The sensor data converter scaler 204 converts the numeric string to a sensor data value having a physical unit for that sensor type. For example, the sensor data value may have a physical unit of ppm or percentage for air quality parameters or degree Celsius for temperature. The converted sensor data is then calibrated using the calibration data for that sensor. The raw sensor data and the calibrated sensor data are then stored in the sensor data database 108.

In some embodiments, the sensor data calibration is performed using the calibration data for that sensor and also the temperature and humidity values taken at the sensor node concurrently with the sensor reading or within the same time frame as the sensor reading. Furthermore, in some cases, sensor data calibration also uses the air pressure value in addition to the temperature and humidity values. For example, the current temperature, humidity and pressure values can be retrieved from the sensor data database 108 as the sensor data for temperature, humidity and pressure is also continuously received and processed and stored in the sensor data database. In other embodiments, other auxiliary data may also be used in the sensor data calibration process. In some embodiments, the calibration of the sensor data is performed using aggregated or summarized temperature and/or humidity data for the previous minute or previous 10 minutes. The aggregated or summarized temperature and humidity data may be retrieved from the sensor data database or from another local storage. The generation of aggregated and summarized sensor data values will be described in more detail below. By incorporating temperature, humidity, pressure, and other factors experienced by the sensors into the calibration process, the accuracy of the sensor calibration can be greatly improved.

The sensor data aggregator 208 in the data processor 104 generates aggregated sensor data for each sensor over one or more predetermined time intervals. In some embodiments, the sensor data aggregator 208 performs initial sensor data aggregation using the calibrated sensor data from the sensor data converter scaler 204. The sensor data aggregator 208 aggregates sensor data at one or more low aggregation levels using the calibrated sensor data. For example, raw sensor data may be received from the sensors in the field on a 1-second interval and processed by the sensor data converter scaler 204 at the 1-second interval. The sensor data aggregator 208 may aggregate the calibrated sensor data for each sensor over one or more low-level time intervals, such as a 1-minute interval and/or a 5-minute interval. The aggregated sensor data may first be stored in a temporary storage in the data processor 104, such as recent reading cache memory 212, and then sent to the sensor data database 108 for storage at a regular time interval.

In embodiments of the present invention, the sensor data aggregator 208 in the data processor performs low level sensor data aggregation. Meanwhile, the sensor data aggregator 112 performs multi-level aggregation as described above to generate aggregated sensor data at one or more time intervals. More specifically, the sensor data aggregator 112 performs high-level sensor data aggregation using the low-level aggregated sensor data generated by the sensor data aggregator 208 and stored in the sensor data database 108. The operation of the sensor data aggregator 112 is agnostic to the operation of the sensor data aggregator 208 in the data processor. Each sensor data aggregator generates aggregated sensor data at the prescribed time intervals and stores the aggregated sensor data at the sensor data database 108.

The metadata updater module 210 detects from the sensor data message changes in the system configuration or base unit configuration. For example, a base unit may be installed with a new controller module or new sensors. The metadata updater module 210 detects for configuration changes and sends configuration updates to the metadata database 106.

In embodiments of the present invention, the data processor 104 further includes a recent reading cache memory 212. Recent reading cache memory 212 stores summarized sensor data to enable quick query of frequently requested sensor data. The summarized data includes sensor data associated with common or frequently requested queries, such as last hour of sensor data. With the recent reading cache memory 212 storing summarized sensor data, the summarized sensor data can be accessed quickly without requesting the data from the sensor data database 108. The recent reading cache memory 212 is optional and may be omitted in some embodiments of the present invention. However, in embodiments of the present invention, the recent reading cache memory 212 provides summarized sensor data which may be advantageously applied to improve the sensor data calibration process.

In particular, as raw sensor data is being received, the sensor data converter scaler 204 processes the raw sensor data by converting the raw sensor data to a physical unit and then calibrating the converted sensor data. As described above, the sensor data calibration can be more accurate in some cases when the temperature and/or humidity and/or pressure and/or other conditions experienced by the sensor at the sampling time is taken into consideration in the calibration computation. In some embodiments, the sensor data converter scaler 204 may query the sensor data database 108 for the recent temperature and/or humidity and/or pressure data, such as requesting aggregated temperature and/or humidity data for a recent time period from the sensor data database 108. However, database query tends to be slow and increased number of queries to the database is sometimes undesirable. Thus, in embodiments of the present invention, the recent reading cache memory 212 stores summarized temperature data, summarized humidity data, summarized pressure data, and/or other factors, for a given recent time period, such as last 10 minutes or most recent 60 minutes. The time period can be variable and adjusted by the data collection engine to store the desired amount of recent data in the cache memory. The summarized temperature, humidity, pressure data, and/or other factors, are stored in the cache memory 212. In this manner, the sensor data converter scaler 204 can retrieve the recent temperature and humidity data from the recent reading cache memory 212 quickly when needed for calibration calculation. The sensor data calibration process at the data processor can be run more efficiently. In other embodiments, the recent reading cache memory 212 also store recent sensor data readings for removing sensor cross-sensitivity during calibration.

Sensor diagnostics module 214 processes the diagnostic data that may be sent in the sensor data message by a sensor. The diagnostic data may be sent periodically to enable the system to keep track of the health of the sensor and/or the base unit. Sensor diagnostics module 214 processes the diagnostic data and provides the data to the appropriate system management modules.

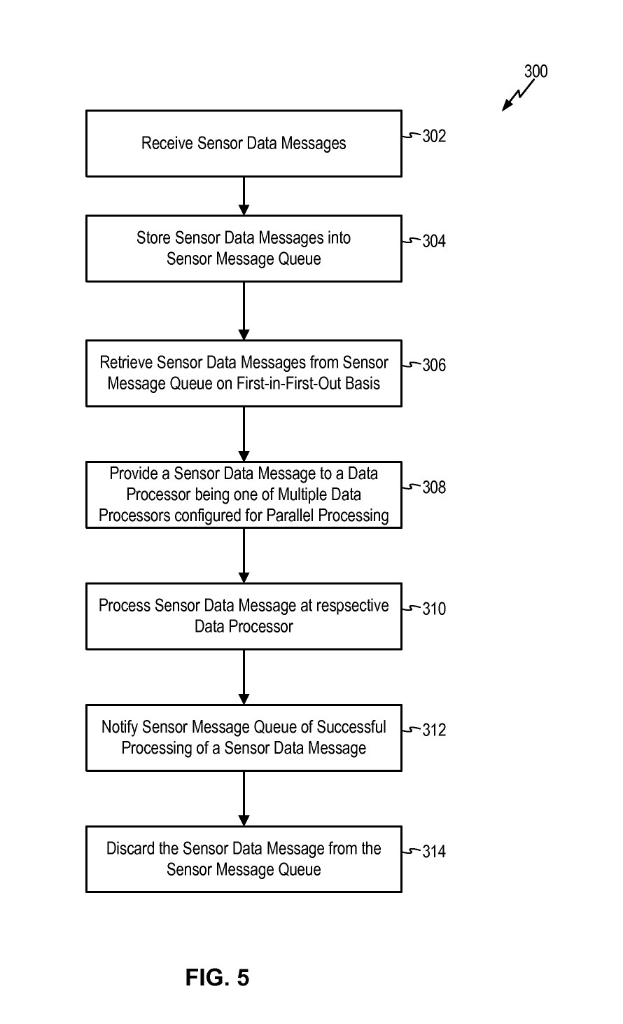

FIG. 5 is a flowchart illustrating a sensor data processing method in embodiments of the present invention. Referring to FIG. 5, a sensor data processing method 300 may be implemented in the centralized backend system 102 of the distributed sensor system 100 in FIG. 1 to collect and process sensor readings generated by the sensors that are deployed in the installation. At 302, the sensor data processing method 300 receives sensor data messages as each sensor in the field reports its sensor reading at each reporting interval. At 304, the sensor data processing method 300 stores the incoming sensor data messages into the sensor message queue. At 306, the sensor data processing method 300 retrieves the sensor data messages from the sensor message queue on a first-in-first-out basis. At 308, the sensor data processing method 300 provides a sensor data message to a data processor being one of multiple data processors configured for parallel processing. At 310, the sensor data processing method 300 processes the sensor data message at the data processor. At 312, after successful completion of the processing of a sensor data message, the sensor data processing method 300 notifies the sensor message queue of the completion status. At 314, the sensor data processing method 300 discards the sensor data message from the sensor message queue.

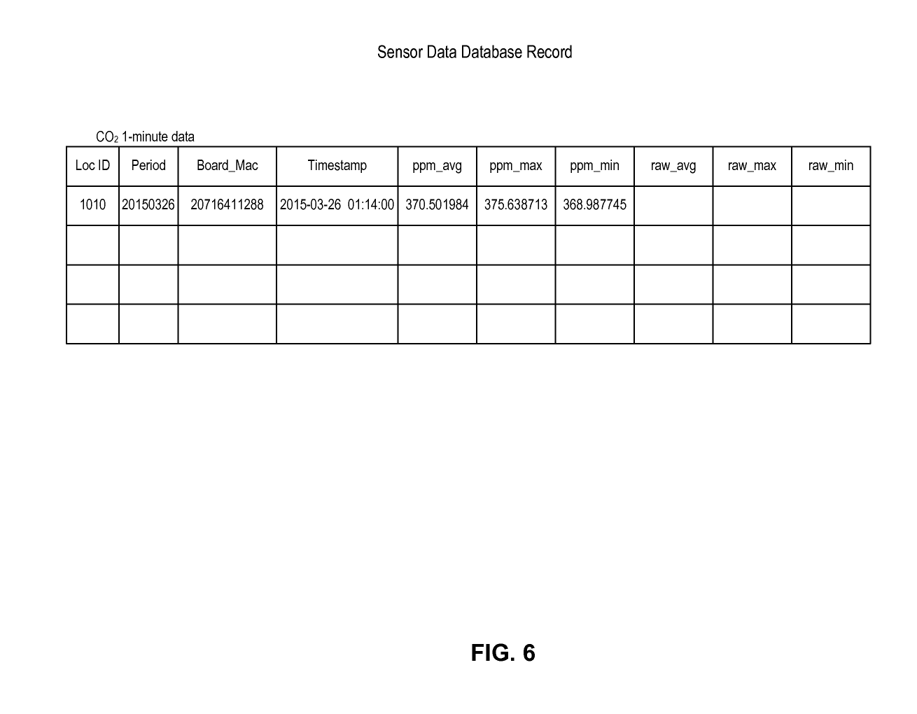

FIG. 6 illustrates an example sensor data record in the sensor data database in embodiments of the present invention. Referring to FIG. 6, a sensor data record in the sensor data database for the carbon dioxide 1 minute data is shown. The sensor data record includes the location ID, the time period (Period), the sensor board ID (or MAC address), and the timestamp of the sensor data. The sensor data record includes the converted and calibrated sensor data including the average, maximum and minimum values in the physical unit of measurement for carbon dioxide. In the present example, the calibrated sensor data is stored in the unit of ppm (parts per million). The sensor data record further includes the raw sensor data, including the average, maximum and minimum raw sensor data values.

Metadata Database

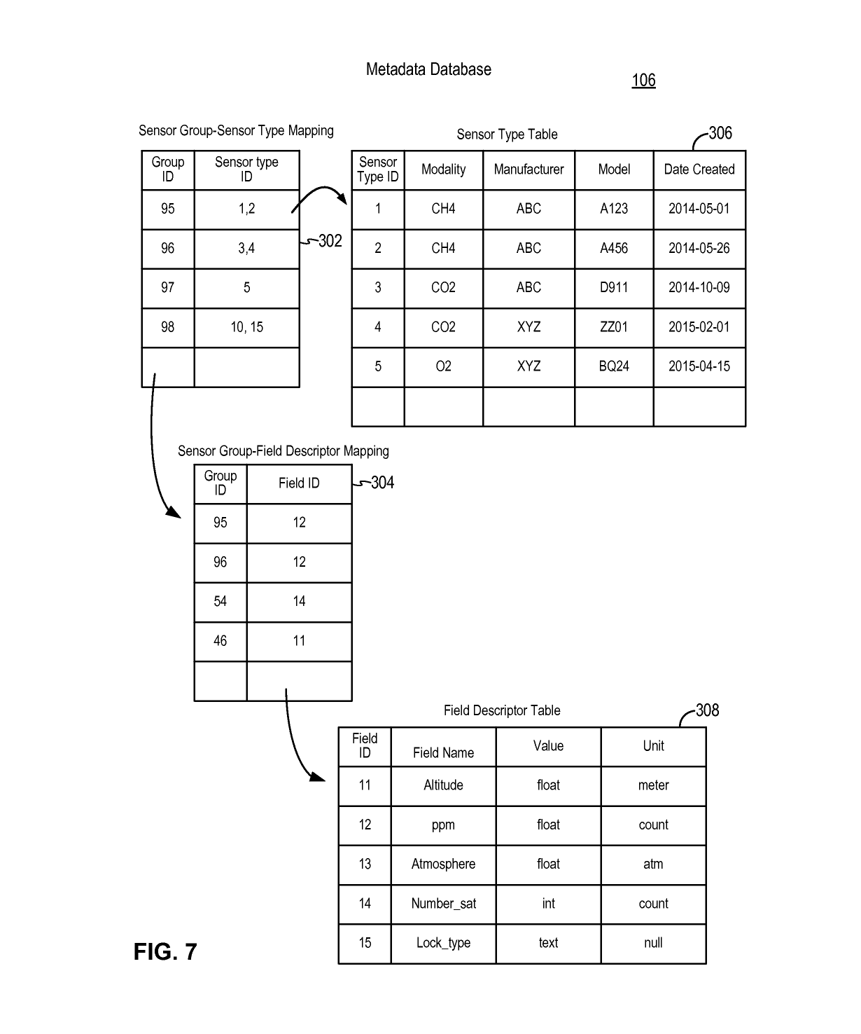

As described above, the sensor data processing system of the present invention implements a generically configured and metadata driven system. The sensor data processing system uses generic data structure for the sensor data messages and uses metadata tables stored in the metadata database to define and configure the sensor data database. FIG. 7 illustrates a configuration of metadata tables in the metadata database to enable the generically configured and metadata driven system in embodiments of the present invention. Referring to FIG. 7, the metadata database 106 in the sensor data processing system includes a configuration of metadata tables that enable generic definition of the data structure used for the sensor data messages and also used to define the data tables in the sensor data database.

In embodiments of the present invention, the metadata database 106 includes a configuration of metadata tables that are cross-referenced to enable the data fields of the sensor data message to be generically defined. A sensor group-sensor mapping table 302 defines a listing of sensor groups and the associated sensor types. The sensor groups are identified by a Group ID (e.g. 95, 96, 97, 98). A sensor group is used to define a set of data field description for the sensor data. In particular, a sensor group-field descriptor mapping table 304 is used to associate each sensor group to an associated field descriptor identifier, referred to as a Field ID. A field descriptor table 308 includes a listing of the Field ID and the associated field descriptions.

In the present embodiment, the field descriptor table 308 includes the following field descriptions: “field name” which denotes the sensor parameter associated with the sensor data, “value” which identifies the sensor parameter having a floating point value, an integer value or a text string, and “unit” which defines the physical unit of the sensor parameter. For example, a field ID of “11” is associated with the parameter “altitude,” has a floating point value and a unit of meter; a field ID of “12” is associated with the parameter “parts-per-million,” has a floating point value and a unit of count; a field ID of “13” is associated with the parameter “atmospheric pressure,” has a floating point value and a unit of “atm”; and a field ID of “14” is associated with the parameter “saturation number,” has an integer value and a unit of count.

As thus configured, the field descriptor table 308 includes a list of field descriptors (Field ID) which defines the field descriptions for various sensor parameters, such as temperature, ppm, concentration, pressure, humidity, and their related field parameters, such as the type of value and the type of physical unit. The field descriptors can then be used to associate with sensor groups through the sensor group-field descriptor mapping table 304. Finally, the sensor groups can be used to associate with different sensors through the sensor group-sensor mapping table 302.

The sensor type table 306 defines a list of sensors, identified by the sensor ID (e.g. 1, 2, 3, 4, 5, etc.) and the associated identifying information. Each sensor ID is defined by the sensor modality, the manufacturer of the sensor and the model number of the sensor. In some cases, a data field of the created date may be also be included to record the date the sensor ID is created. In this manner, each type of sensors used in the distributed sensor system is uniquely identified by a sensor ID. For example, a sensor ID of “1” refers to a CH₄ sensor manufactured by ABC and having a model number of A123, and a sensor ID of “2” refers also to a CH₄ sensor manufactured by ABC but having a model number of A456. A sensor ID of “3” refers to a CO₂ sensor also manufactured by ABC but having a model number of D911. Finally, a sensor ID of “4” refers to a CO₂ sensor manufactured by XYZ but having a model number of ZZ01.

Through the sensor group-sensor type mapping table 302, the sensor types are associated with respective sensor groups which define the data fields for the sensor type.

Accordingly, the sensor group ID 95 is related to sensor type 1 and 2 (CH₄ sensors). The sensor type may be of different models but share the same data fields and so can be grouped with the same sensor group. The sensor group ID 95 defines a field ID of 12 which defines the data fields to be ppm, a floating point value and a unit of count.

By using the metadata tables 302, 304, 306 and 308, each type of sensor in the distributed sensor system can be uniquely identified and uniquely described. More importantly, changes to the distributed sensor system, such as the addition of new sensors, can be accomplished by modifying the metadata tables. For example, a new sensor can be added to the sensor type table 306 and associated with the appropriate sensor group and the new sensor is then completely defined in the system. The database factory described above parses the metadata tables periodically to detect changes to the sensor system. When changes are detected, such as the addition of a new sensor type, the database factory reconfigures the sensor data database accordingly so that the sensor data database is ready to receive sensor data from the new sensors.

With the sensor data processing system generically configured, the sensor data processing system of the present invention can be easily adapted to process sensor data generated by sensors of a wide range of sensor modalities. In some embodiment, the sensor data processing system is configured to process sensor data having these sensor modalities: CO₂, O₂, CO, HCHO, NO₂, NO, CH₄, SO₂, O₃, sound, light, temperature, humidity, vibration, pressure, black carbon, position, wind direction, wind speed, heading, ultrafine particles, particulate matter up to 10 micrometers in diameter (PM₁₀) and particulate matter less than 2.5 micrometers in diameter (PM_{2.5}).

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method in a distributed sensor system, the distributed sensor system including a plurality of spatially distributed sensor nodes in communication with a data network, each sensor node comprising at least one sensor configured to measure an air quality parameter or an environmental parameter, the method comprising:
providing a sensor data database including data tables for storing raw sensor data and processed sensor data;
providing a metadata database including metadata tables used to construct definitions for the data tables in the sensor data database;
receiving sensor data messages sent by one or more sensors in the sensor nodes of the distributed sensor system, each sensor data message including generically defined data fields adapted for reporting sensor data associated with different types of sensors;
storing the sensor data messages in a message queue;
providing the sensor data messages in the message queue on a first-in-first-out basis to a plurality of data processors having a parallel processing configuration, where each sensor data message is provided to one data processor;
maintaining the sensor data messages sent out to one or more data processors in the message queue;
processing, at each data processor, sensor data contained in the sensor data message;
storing raw and processed sensor data in the sensor data database;
in response to a sensor data message having completed processing by a data processor, discarding the sensor data message from the message queue;
generating, at the data processors, configuration update information based at least in part on the sensor data messages;
providing, by the data processors, the configuration update information to the metadata database to modify the metadata tables, there by modifying the definitions for the data tables for the sensor data database; and
providing, on a given time interval, modified table definitions to the sensor data database to modify or create data tables for storing raw or processed sensor data.

2. The method of claim 1, wherein processing, at each data processor, sensor data contained in the sensor data message comprises:
decoding the sensor data messages to obtain decoded sensor data including sensor identifying information and sensor readings;
converting the sensor readings to sensor data values having physical units associated with the respective types of sensors that generated the sensor readings;
calibrating each sensor data value using calibration data associated with the sensor identified by the sensor identifying information accompanying the sensor readings; and
generating configuration update information based at least in part on the sensor data message being processed by the data processor.

3. The method of claim 1, wherein processing, at each data processor, sensor data contained in the sensor data message comprises:
processing the sensor data using calibration data stored in the metadata database for the respective sensor.

4. The method of claim 1, wherein processing, at each data processor, sensor data contained in the sensor data message further comprises:
storing sensor data values in a local memory at the data processor for a first time interval;
aggregating the sensor data values for the same sensor processed by the data processor over the first time interval to generate aggregated sensor data values for the sensor; and
storing the aggregated sensor data values in the sensor data database.

5. The method of claim 1, wherein processing, at each data processor, sensor data contained in the sensor data message further comprises:
processing diagnostics data in the sensor data message to determine an operation condition of the sensor node.

6. The method of claim 1, wherein providing, on a given time interval, modified table definitions to the sensor data database to modify or create data tables for storing raw or processed sensor data further comprises:
querying the sensor metadata database periodically to detect for changes in one or more of the metadata tables; and
in response to a detected change in a metadata table, sending instructions to the sensor data database to modify or create the data tables in the sensor data database.

7. The method of claim 1, wherein processing, at each data processor, sensor data contained in the sensor data message further comprising:
processing the sensor data messages using multiple data processors in parallel, each data processor processing an entire sensor data message to completion of processing.

8. The method of claim 1, wherein providing a metadata database including metadata tables used to construct definitions for the data tables in the sensor data database comprises:
providing a plurality of metadata tables, each metadata table being cross-referenced with at least one other metadata table, the plurality of metadata tables including a first metadata table uniquely defining the types of sensors in the distributed sensor system, a second metadata table defining sensor groups with associated field descriptions, and a third metadata table associating one or more types of sensors with a sensor group.

9. A sensor data processing system in a distributed sensor system, the distributed sensor system including a plurality of spatially distributed sensor nodes in communication with a data network, each sensor node comprising at least one sensor configured to measure an air quality parameter or an environmental parameter, the sensor data processing system comprising:
a message queue to store sensor data messages sent by one or more sensors in the sensor nodes of the distributed sensor system, each sensor data message including generically defined data fields adapted for reporting sensor data associated with different types of sensors;
a plurality of data processors configured for parallel processing, the data processors to receive sensor data messages from the message queue on a first-in-first-out basis, each data processor processing raw sensor data contained in the sensor data message and generating processed sensor data;
a sensor data database in communication with the plurality of data processors to store raw sensor data and processed sensor data in data tables; and
a sensor metadata database including metadata tables used to construct definitions for the data tables in the sensor data database,
wherein the message queue provides the sensor data messages to the plurality of data processors on a first-in-first-out basis and maintains the sensor data messages sent out to one or more data processors in the message queue until the sensor data messages have completed processing at the data processors, and in response to a sensor data message having completed processing by a data processor, discarding the sensor data message from the message queue; and
wherein the data processors generate configuration update information based at least in part on the sensor data messages and provide the configuration update information to the metadata database to modify the metadata tables, there by modifying the definitions for the data tables for the sensor data database; and the sensor data database receiving, on a given time interval, modified table definitions to modify or create data tables for storing raw or processed sensor data.

10. The sensor data processing system of claim 9, wherein each of the plurality of data processors is configured to decode the sensor data messages to obtain decoded sensor data including sensor identifying information and sensor readings, to convert the sensor readings to sensor data values having physical units associated with the respective types of sensors that generated the sensor readings, to calibrate each sensor data value using calibration data associated with the sensor identified by the sensor identifying information accompanying the sensor readings, and to generate configuration update information based at least in part on the sensor data message being processed by the data processor.

11. The sensor data processing system of claim 9, wherein each of the plurality of data processors is further configured to processing the sensor data using calibration data stored in the metadata database for the respective sensor.

12. The sensor data processing system of claim 9, wherein each of the plurality of data processors is further configured to store sensor data values in a local memory at the data processor for a first time interval, to aggregate the sensor data values for the same sensor processed by the data processor over the first time interval to generate aggregated sensor data values for the sensor, and to store the aggregated sensor data values in the sensor data database.

13. The sensor data processing system of claim 9, wherein each of the plurality of data processors is further configured to process diagnostics data in the sensor data message to determine an operation condition of the sensor node.

14. The sensor data processing system of claim 9, wherein the sensor data processing system further comprises a database factory module configured to query the sensor metadata database periodically to detect for changes in one or more of the metadata tables and in response to a detected change in a metadata table, the database factory module sends instructions to the sensor data database to modify or create the data tables in the sensor data database.

15. The sensor data processing system of claim 9, wherein each of the plurality of data processors is configured to process the sensor data messages in parallel, each data processor processing an entire sensor data message to completion of processing.

16. The sensor data processing system of claim 9, wherein the metadata database comprises a plurality of metadata tables, each metadata table being cross-referenced with at least one other metadata table, the plurality of metadata tables including a first metadata table uniquely defining the types of sensors in the distributed sensor system, a second metadata table defining sensor groups with associated field descriptions, and a third metadata table associating one or more types of sensors with a sensor group
